(19) United States
(12) Patent Application Publication
(10) Pub. No.: US 2004/0254455 A1
(43) Pub. Date: Dec. 16, 2004

(54) Magneic switch for use in a system that includes an in-vivo device, and method of use thereof

(76) Inventor: Gavriel J. Iddan, Haifa (IL)

Correspondence Address: EITAN, PEARL, LATZER & COHEN ZEDEK LLP, 10 ROCKEFELLER PLAZA, SUITE 1001, NEW YORK, NY, 10020, US

(21) Appl. No.: 10/811,013

(22) Filed: Mar. 29, 2004

Related U.S. Application Data
(63) Continuation-in-part of application No. 10/130,326, filed on May 15, 2002.
(60) Provisional application No. 60/457,592, filed on Mar. 27, 2003.

(51) Int. Cl. A61B 6/00

(52) U.S. Cl. 600/424; 600/476; 128/899; 128/903

(57) ABSTRACT

A switch such as a normally closed MEMS switch may be used in an in-vivo device to alter a mode or operation; for example, to disconnect the power unit from electrical circuitry during storage of the in-vivo device by introducing a magnetic field in the proximity of the in-vivo device. The in-vivo de vice may be powered by removing the magnetic field upon use. Other switches, such as normally open switches, or switches including multiple sets of leads, having multiple functionality, may be used. A packaging generating a magnetic field may operate the switch when the device is in the packaging.

### CROSS REFERENCE TO RELATED APPLICATIONS

The present application claims benefit from prior U.S. provisional application Ser. No. 60/457,592, filed 27 Mar. 2003, entitled “Normally Closed MEMS Switch and Device using Switch”, incorporated herein by reference in its entirety. The present application is also a continuation-in-part of U.S. patent application Ser. No. 10/130,326, filed May 15, 2002, entitled “Method For Activating An Image Collecting Process,” and incorporated by reference herein.

### FIELD OF THE INVENTION

The present invention relates to a magnetic operated switch such as a MEMS switch and to an in-vivo device, system and method that uses such switches.

### BACKGROUND OF THE INVENTION

Devices and methods for performing in-vivo imaging of passages or cavities within a body, and for gathering information other than or in addition to image information (e.g., temperature information, pressure information), are known in the art. Such devices may include, inter alia, various endoscopic imaging systems and devices for performing imaging in various internal body cavities.

An in-vivo imaging device may include, for example, an imaging system for obtaining images from inside a body cavity or lumen, such as the GI tract. The imaging system may include, for example, an illumination unit, an optical system, and possibly a transmitter and antenna. Other types of in-vivo devices exist, such as endoscopes that may not require a transmitter, and devices performing functions other than imaging.

Various systems and devices, for example, in-vivo imaging devices, may require that certain electrical circuits be closed during most of the time of their operation. Some switches, for example, normally open reed switches may require that the operating device be in the proximity of a magnetic field to keep the switch closed. A ballast magnet may be added to keep the switch closed, however, it may be inefficient and/or inconvenient to maintain the switch in proximity to the magnetic field, or to generate and/or maintain the magnetic field required to keep the switch closed. Size may be an additional factor in an m-vivo device. Reed switches may be for some in-vivo devices too voluminous.

Micro-Electro-Mechanical Systems (MEMS) devices are typically extremely small mechanical devices. A typical MEMS switch may include two or more terminals, typically made from or including a soft ferromagnetic material, sealed inside a plastic, glass, or other container containing vacuum or gas. One of the terminals may be connected to a reed-like elastic element that may bend in reaction to a magnetic field applied thereto. Other structures and other suitable materials may be used in a MEMS switch.

Typically MEMS switches may be normally open, whereby the two terminals are generally parallel, i.e., not in contact, and partly overlap each other. When the reed switch is in the presence of a magnetic field, one or more of the terminals may bend and touch the other terminal, thereby closing an electrical circuit. In some such switches, a small contact surface area may limit the current flow that may be passed through the switch.

### SUMMARY OF THE INVENTION

Various embodiments of the present invention provide a switch such as a MEMS switch that may be used, for example, in an in-vivo device such as, for example, an in-vivo imaging device. In one embodiment of the present invention, the in-vivo device may be part of a system and may record, process, and display data transmitted by the in-vivo device.

According to embodiments of the present invention, an in-vivo device may include one or more MEMS switches, for example, a MEMS reed switch to connect and/or disconnect a battery or other power supply or power unit while the in-vivo imaging device is in use and/or in storage or dormant.

Embodiments of the present invention further provide a method of using a normally closed MEMS switch in a circuit of an in-vivo imaging device. Such a MEMS switch may be used in applications other than an in-vivo device.

In one embodiment of the invention, an in-vivo device includes at least a sensor and a MEMS switch. The sensor may be for example an imager. The in-vivo device may be for example a swallowable capsule. The MEMS switch may be for example operable to alter the mode of the device, possibly in response to a magnetic field. The device may include a transmitter. The MEMS switch may be for example a normally closed MEMS switch. The MEMS switch may include for example a first ferromagnetic conductive terminal; a flexible ferromagnetic conductive terminal; and a non-magnetic conductive terminal; wherein the first ferromagnetic conductive terminal and the non-magnetic conductive terminal are electrically isolated.

In one embodiment of the invention, an in-vivo device includes at least a sensor; and a switch, the switch including at least a first ferromagnetic conductive terminal; a flexible ferromagnetic conductive terminal; and a non-magnetic conductive terminal; wherein the first ferromagnetic conductive terminal and the non-magnetic conductive terminal are electrically isolated. The switch may be for example a MEMS switch. The sensor may be for example an imager. The in-vivo device may be for example a swallowable capsule. The switch may be for example operable to alter the mode of the devices possibly in response to a magnetic field.

In one embodiment of the invention, a system for in-vivo imaging includes at least an in-vivo device including at least a sensor and a MEMS switch; and an external control device, the external control device including at least a magnetic field source producing a magnetic field sufficient to operate the switch. The sensor may be for example an imager. The system may include a controller to receive data relating to an in-vivo condition and, in response, operate the magnetic field source, and/or to determine the m-vivo condition. The condition may be the location of the in-vivo device. The operation of the switch may alter the operation of the in-vivo device. Altering the operation may include stopping the operation of a component of the in-vivo device. The MEMS switch may include a first ferromagnetic conductive terminal; a flexible ferromagnetic conductive terminal; and a non-magnetic conductive terminal; wherein the first ferromagnetic conductive terminal and the non-magnetic conductive terminal are electrically isolated. The device may be a swallowable capsule.

In one embodiment of the invention, a MEMS switch includes for example a first ferromagnetic conductive terminal; a flexible ferromagnetic conductive terminal; and a non-magnetic conductive terminal; wherein the first ferromagnetic conductive terminal and the non-magnetic conductive terminal are electrically isolated. The switch may for example include a housing, wherein the housing encloses: the first ferromagnetic conductive terminal; the flexible ferromagnetic conductive terminal; and the non-magnetic conductive terminal. The switch may enclose a dry gas. The first ferromagnetic conductive terminal and the flexible ferromagnetic conductive terminal may be coated with a non-stiction layer, and a non-conductive layer may be included, possibly coated with an insulating layer. The switch may include for example a common lead; and a normally open lead. The switch may include for example a common lead; and a normally closed lead.

In one embodiment of the invention, a method includes for example operating an in-vivo device in a body, the in-vivo device including at least: a sensor; and a normally closed magnetically operable switch; and causing a magnetic field to operate the switch. The magnetic field source may be operated by a receiving system receiving data from the in-vivo device. The method may include receiving in-vivo data from the in-vivo device and, in response, operating the magnetic field. Causing a magnetic field to operate the switch may include adding a magnetic field. The method may include altering a mode of the in-vivo device. The method may include receiving at a receiving system image data transmitted from the in-vivo device. The in-vivo device may be a swallowable capsule. The switch may be a MEMS switch.

One embodiment of the invention may include an in-vivo sensing system including for example an in-vivo sensing device including at least: a normally closed magnetic switch; and a sensor. The switch may be for example a MEMS switch. The sensor may be for example an imager. The device may include at least a transmitter.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features and advantages thereof, may best be understood by reference to the following detailed description when read with the accompanied drawings in which:

FIG. 1A is a schematic illustration of an in-vivo imaging system in accordance with embodiments of the present invention;

FIG. 1B is a schematic illustration of an in-vivo imaging system with a sensor in accordance with an embodiment of the present invention;

FIG. 2A is a schematic illustration of a MEMS switch in a closed, non-activated mode in accordance with embodiments of the present invention;

FIG. 2B is a schematic illustration of the MEMS switch of FIG. 2A in an activated, open mode in accordance with embodiments of the present invention;

FIG. 3 is a flow chart describing a method for keeping an in-vivo device dormant according to an embodiment of the present invention;

FIG. 4 describes a method for altering the mode or operation of an in-vivo device according to an embodiment of the present invention; and

FIG. 5 depicts an in-vivo device and a package or container for holding the device, according to an embodiment of the present invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE INVENTION

In the following description, various aspects of the present invention will be described. For purposes of explanation, specific configurations and details are set forth in order to provide a thorough understanding of the present invention. However, it will also be apparent to one skilled in the art that the present invention may be practiced without the specific details presented herein. Furthermore, well-known features may be omitted or simplified in order not to obscure the present invention.

Embodiments of the system and method of the present invention are typically used in conjunction with an in-vivo sensing system or device such as described in U.S. Pat. No. 5,604,531 to Iddan et al. and/or in International Application number WO 01/65995 entitled “A Device And System For In-Vivo Imaging”, published on 13 Sep. 2001, both of which are assigned to the common assignee of the present invention and both of which are hereby incorporated by reference. However, a device, system and method according to various embodiments the present invention may be used with any in-vivo device. Alternate embodiments of the system and method of the present invention may be used with other devices, non-imaging and/or non-in-vivo devices.

Embodiments of the in-vivo device may typically be autonomous and may typically be self-contained. For example, the in-vivo device may be a capsule or another unit where all the components may be substantially contained within a container or shell, and where the in-vivo device may not require any wires or cables to, for example, receive power or transmit information. The in-vivo device may communicate with an external receiving and display system to provide display of data, control, or other functions. For example, power may be provided by an internal battery or a wireless receiving system. Other embodiments may have other configurations and capabilities. For example, components may be distributed over multiple sites or units. Control information may be received from an external source.

A system according to some embodiments of the invention includes an in-vivo sensing device transmitting information (e.g., images or other data) to a data receiver and/or recorder possibly close to or worn on a subject. A data receiver and/or recorder may take other suitable configurations. The data receiver and/or recorder may transfer the received information to a larger computing device, such as a workstation or personal computer, where the data may be further analyzed, stored, and/or displayed to a user. In other embodiments, each of the various components need not be required; for example, an internal device may transmit or otherwise transfer (e.g., by wire) information directly to a viewing or processing system.

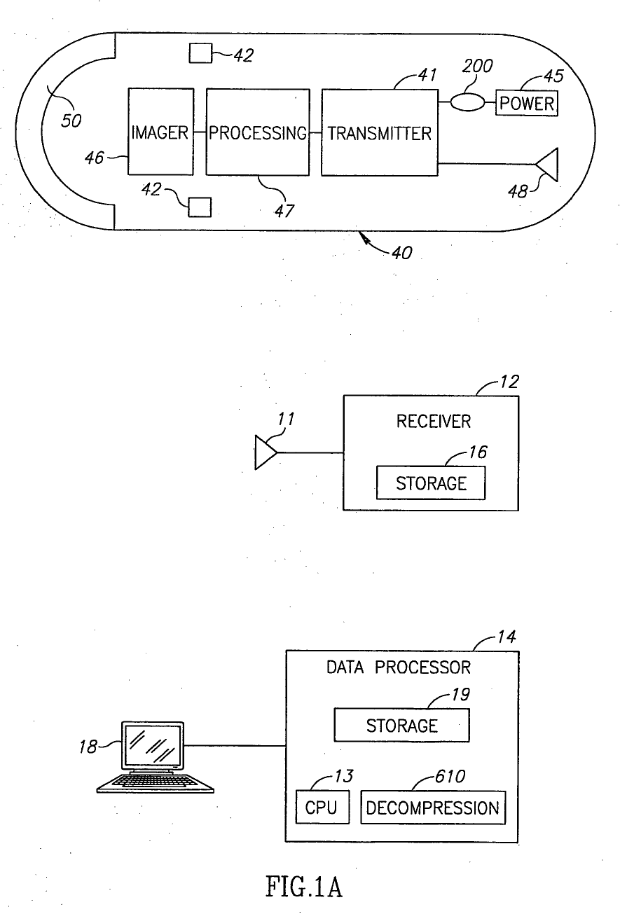

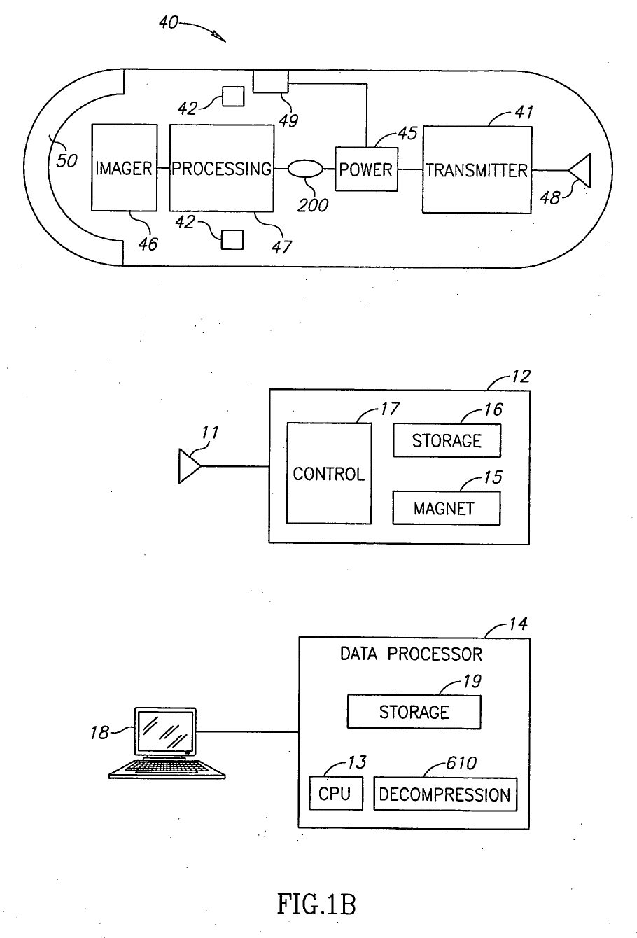

Reference is made to FIG. 1A, which shows a schematic diagram of an in-vivo imaging system according to one embodiment of the present invention. In one embodiment, a device 40 may be, for example, a swallowable capsule capturing images, but may also be another device that may collect information other than or in addition to image information. Typically, device 40 may include at least one sensor such as, for example, an imager 46, for capturing images, a processing chip or circuit 47 that processes the signals generated by the imager 46, and one or more illumination sources 42, for example one or more white LEDs or any other suitable light source, for illuminating the body lumen. An optical system 50, including, for example, one or more optical elements (not shown), such as one or more lenses or composite lens assemblies (not shown), one or more suitable optical filters (not shown), or any other suitable optical elements (not shown), may aid in focusing reflected light onto the imager 46 and performing other light processing. Processing chip 47 need not be a separate component; for example, processing or a processing chip may be integral to the imager 46, and may not be needed. The sensor may be another type of sensor, such as a temperature sensor, a pH sensor, a pressure sensor, or other suitable sensor.

Device 40 typically may include a transmitter 41, for transmitting image and possibly other information (e.g., control information) to a receiving device 12. The transmitter 41 may typically be an ultra low power radio frequency (RF) transmitter with high bandwidth input, possibly provided in chip scale packaging. The transmitter 41 may transmit via an antenna 48. The transmitter 41 may also include circuitry and functionality for controlling the device 40. Typically, the device 40 may include a power source or a power unit 45, such as one or more batteries. For example, the power source 45 may include silver oxide batteries, lithium batteries, or other electrochemical cells having a high energy density, or the like. Other suitable power sources may be used; for example power source 45 may be a unit designed to receive power from an external source.

In one embodiment, the imager 46 may be a complementary metal oxide semiconductor (CMOS) imaging camera. Such a CMOS imager may typically be an ultra low power imager and may be provided in chip scale packaging (CSP). Other types of CMOS imagers may be used. In another embodiment, another imager may be used, such as a CCD imager, or another suitable imager.

In an embodiment of the present invention, device 40 may include a switch such as a switch 200, for example a normally closed MEMS switch or other suitable switch, that may open in the presence of a magnetic field, the structure and functionality of which are described in detail herein. Switch 200 may be used, for example, to disconnect power source 45 during storage and/or during periods of time at which it is desired to keep device 40 dormant. For example, in an embodiment of the present invention, switch 200 may be connected to electrical circuit that includes power source 45, and since switch 200 is normally closed, it may keep the electrical circuit closed without the presence of a magnetic field. Additionally or alternatively, at times when it is desired that the device 40 not be operational, for example, when the device 40 may be in storage, the device 40 may be placed in proximity to a magnetic field and thus switch 200 may open and may disconnect any suitable electrical circuit, for example, a circuit that includes power source 45.

It will be appreciated by persons skilled in the art that switch 200 may be used for various functionalities within the device 40, for example, to bring an electrical circuit within the device 40 from a closed mode to an open mode, or vice versa. Additionally or alternatively, switch 200 may be used to change a property or mode of the device 40 and/or its functionality, for example, switch 200 may be electrically connected to a processing chip 47, or to processing circuit on the imager 46 and its operation may for example change the image capturing rate of the device 40. The operation of switch 200 may control a circuit that in turn controls the operation or on/off state of the device 40. It is noted that, in embodiments of the present invention, device 40 may include a switch 200 or a plurality of switches 200, to perform one or more of such functionalities, to close and/or open one or more of such electrical circuits, or to connect and/or disconnect one or more power sources similar to power source 45. As described in detail below, actuation of switch 200 into open mode may be achieved, for example, by an external magnet, such as a permanent magnet, a ballast magnet, an external electromagnetic coil, and the like.

Typically, the device 40 may be swallowed by a patient and traverses a patient's GI tract, however, other body lumens or cavities may be imaged or examined. The device 40 may transmit image and possibly other data to components located outside the patient's body, which may receive and process the data. Typically, located outside the patient's body in one or more locations, are a receiver 12, typically including an antenna or antenna array 11, for receiving image and possibly other data from device 40, a receiver storage unit 16, for storing image and other data, a data processor 14, a data processor storage unit 19, an optional data decompression module 610 for decompressing compressed data, and a display, such as for example an image monitor 18, for displaying, inter alia, the images transmitted by the device 40 and recorded by the receiver 12. Receiver 12 may include a controller 17 or processor which may, for example, control data collection, control the transmission of signals to device 40, etc. Typically, the receiver 12 and receiver storage unit 16 may be small and portable, and may be worn on the patient's body during recording of the images. Typically, data processor 14, data processor storage unit 19 and monitor 18 may be part of a personal computer or workstation, which may include standard components such as, for example, a controller 17 or processor 13 (e.g. CPU, or other processor), a memory (e.g., storage 19, or other memory), a disk drive, and input-output devices, although alternate configurations are possible. In alternate embodiments, the data reception and storage components may be of another configuration. Further, image and other data may be received in other manners, by other sets of components. Typically, in operation, image data is transferred to the data processor 14, which, in conjunction with processor 13 and software, stores, possibly processes, and displays the image data on monitor 18. Other systems and methods of storing and/or displaying collected image data may be used.

Reference is now made to FIG. 1B, which shows a schematic diagram of an in-vivo imaging system according to another embodiment of the present invention. In device 40, imager 46 may be electrically connected to transmitter 41 and power source 45 via a switch 200 such as a MEMS switch or other suitable magnetically operated switch while sensor 49 may be directly electrically connected to transmitter 41 and/or power source 45. Sensor 49 may be for example, a temperature sensor, pH sensor, pressure sensor, light sensor or other suitable sensor. Sensor 49 may, for example, transmit sensed data via transmitter 41 and antenna 48 to an external receiver 12. A controller (e.g., controller 17, or processor 13) may detect or receive information regarding an in-vivo condition (e.g., the presence of a substance, the position of an in-vivo device) and, in response operate a magnetic field source. For example, receiver 12 may include one or more electromagnetic coil(s) 15 or other suitable unit(s) that may generate or add a magnetic field. Typically, the magnetic field is of sufficient strength to operate a MEMS or other suitable switch, which may be located in an in-vivo device. In one embodiment of the invention, the magnetic field may be initiated or alternatively terminated upon reception of data from sensor 49 or imager 46 corresponding to predetermined values and/or thresholds. Analysis may be performed on data received from sensor 49 or imager 46 to cause such a magnetic field operation—for example, an image analysis detecting the presence of a substance, a change in light levels, etc. A sensor 49 need not be used. Further, a magnetic field generating unit may be separate from the receiver 12. Termination or removal of the magnetic field generated by electromagnetic coil 15 may set off closure of switch 200 which may effect an operational change in device 40 or in components of device 40. For example, imager 46 may become electrically connected with power source 45 and transmitter 41. Other operational changes, such as a change in image capture rate, a change in lighting, etc., may be effected. Various analysis methods may be performed on data received from sensor 49 or imager 46 to cause an external unit such as receiver 12 to turn on or alter a magnetic field, and such a magnetic field may have other suitable effects on device 40. In one embodiment, a change in a magnetic field may cause imager 46 to begin to capture images and/or the transmitter 41 to transmit images.

An external processor, for example located in receiver 12 or in data processor 14, may control a magnetic source to control or operate the in-vivo device 40. For example, a controller may receive data regarding an in-vivo condition (e.g., pressure, pH, location, etc.), or may itself compute the existence of such a condition. An “in-vivo condition” may include the condition of the device 40, such as its motility, location, etc. The controller may then operate the electromagnetic source, which may operate a switch internal to the in-vivo device 40. In response, the operation of the device 40 may be changed. For example, in response to the detection of a location or relative location of device 40, a frame capture rate may be altered. In response to a relative lack of movement of device 40, the device 40 may lie dormant for a time, certain components (e.g., an imager, a transmitter, etc.) may have their operation reduced or stopped. Other combinations of in-vivo or device conditions and mode or operational changes may be used.

In another embodiment of the invention, a magnetic field, or the generation, addition or a removal of a magnetic field, may be implemented in an alternate manner. For example, an external magnet may be physically placed in the vicinity of the device 40. Receiver 12 may indicate reception of data corresponding to predetermined values and/or thresholds with an audio or visual signal and thus prompt a user to remove or alternatively position the external magnet. In other embodiments of the invention, the switch 200 may be positioned in an alternate manner and may be electrically connected to other components or sets of components in device 40. For example, in one embodiment of the invention, a MEMS switch may be used to switch on/off powering to the transmitter 41. As such, transmission may be controlled with the presence and/or absence of a magnetic field. According to other embodiments a MEMS switch could be used to switch on/off illumination sources.

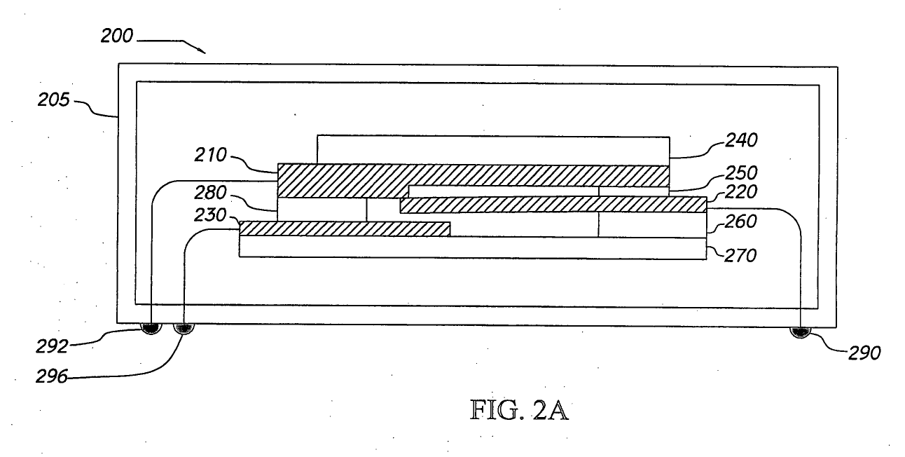

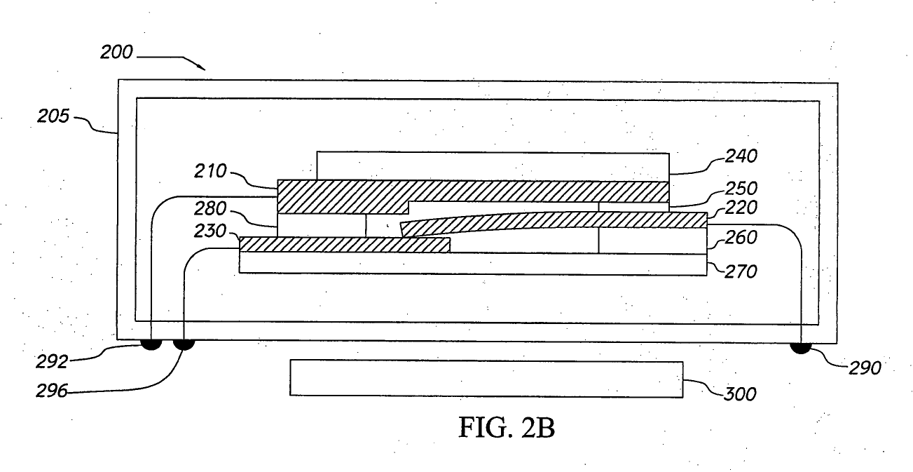

FIG. 2A is a schematic illustration of a MEMS switch 200 in a closed, non-activated mode in accordance with embodiments of the present invention. Switch 200 may include a housing 205, for example a Surface Mount Packaging (SMP), which may enclose terminals 210, 220 and 230, and layers 240, 250, 260, 270 and 280. Other suitable types of packaging may be used, for example other suitable packaging used for other known silicon devices. Materials such as plastic may be used, and other numbers of layers may be used. The housing may be filled with for example a vacuum or a gas such as a dry noble gas, for example Nitrogen or Aragon and for example, be sealed.

Terminal 210 may be formed of a non-magnetic conductive material, such as non-magnetic metal, for example, gold or chromium or any other suitable, conductive material or alloy. Each of terminals 220 and 230 may include a terminal made, for example, of a typically soft, ferromagnetic, conductive material, for example, Permalloy. Terminals 220 and 230 may be coated with a suitable non-stiction layer, which may prevent terminals 220 and 230 from resisting separation after being in contact. Terminal 220 may be a flexible ferromagnetic conductive terminal.

Non-conductive layers 240, 250, 260, 270 and 280 may include MEMS layers made of, for example, silicon, and may be coated for insulation with an insulating layer, for example, silicon oxide or silicon nitride. Materials other than silicon-based materials may be used. Further, other numbers of layers and other structures may be used. For example, the support and spacing provided by layers 260 and 280 may be provided by other structures.

Terminals 210, 220 and 230, as well as layers 240, 250, 260, 270 and 280, may be fabricated in shapes and placed as illustrated schematically in FIG. 2A, such that terminal 220 may have a pre-determined elastic deformation and touch terminal 210 but, when activated by a magnetic field, may not touch terminal 230.

Various leads and combinations of leads may be used. Common lead 290, normally closed lead 292, and normally open lead 296, may be in the form of a ball grid array, a lead frame, or other suitable leads. When connecting common lead 290 and normally closed lead 292 to an electric circuit, the MEMS switch 200 acts as a normally closed switch. When connecting common lead 290 and normally open lead 296 to an electric circuit, the MEMS switch 200 may act as a normally open switch. As such the MEMS switch described in this embodiment of the present invention may serve as both a normally open switch and a normally closed switch. In one embodiment of the invention, leads 290, 292, and 296 may be an integral part of terminals 220, 210, and 230 respectively. Other sets of leads may be used. For example, one of leads 292 and 296 may be omitted to create a switch that is only normally open or normally closed.

Reference is now made also to FIG. 2B, which is a schematic illustration of a MEMS switch 200 in accordance with an embodiment of the present invention, when placed in the proximity of a magnetic field. Switch 200 is shown placed in proximity to magnet 300, causing switch 200 to be switched to an open mode. It is noted that magnet 300 may include any suitable magnet or source of magnetic field, for example, a ballast magnet, a permanent magnet, an electromagnetic coil, and the like. Upon such placement of switch 200 in proximity to magnet 300, terminal 220 and 230 attain magnetic properties and terminal 220 may bend or move away from terminal 210, creating an open position between terminal 220 and terminal 210. Furthermore, although the present invention is not limited in this regard, terminal 220 may bend and touch terminal 230, forming and/or closing an electric circuit and creating a closed position between terminal 220 and terminal 230.

Although the present invention is not limited in this regard, in an embodiment of the present invention, upon placement of switch 200 in proximity to magnet 300, terminals 220 and 230 may form a magnetic “short circuit” of the flux lines generated by the external magnet 300. For example, in an embodiment of the present invention, in order to get minimal magnetic resistance, terminal 220 may bend towards terminal 230, thereby opening the electrical circuit to which terminals 210 and 220 may be connected. Thus, upon applying the magnetic field, terminal 220 and terminal 210 may be in open mode and, in an embodiment of the present invention, terminal 220 and terminal 230 may be in closed mode. It is noted that the same result may be obtained using other sources of magnetic field, in addition to or instead of external magnet 300, for example, using an electromagnetic coil. Further, while a two-way switch is shown, other numbers of terminals may be used.

Although the present invention is not limited in this regard, switch 200 in accordance with embodiments of the present invention may be used, for example, as a part of an electrical circuit that needs to be closed by default without using a magnetic field, e.g., most of the time, and may be opened by a magnetic field when necessary, e.g., a small percentage of the time. Alternatively, switch 200 in accordance with another embodiment of the present invention may be used, for example, as a part of an electrical circuit that needs to be opened by default without using a magnetic field, e.g., most of the time, and may be closed by a magnetic field when necessary, e.g., a small percentage of the time.

During operation, switch 200 in accordance with embodiments of the invention, may be placed and/or connected, for example, in an electrical circuit within an in-vivo device. Upon such placement or connection, the normally closed switch 200 may be in a closed mode. The surface contact area that may be achieved between terminals 220 and 230 may allow for a high current to pass through the switch in the closed mode. Further, while a two-way switch is shown, other numbers of terminals may be used.

Switch 200 may be switched into open mode (or, using a suitable arrangement of terminals, into a closed mode) by adding or increasing a magnetic field. In embodiments of the present invention, this may be performed using an external magnet or any suitable source of magnetic field, for example, electromagnetic coil. In one embodiment, where switch 200 is included in an in-vivo device, the magnetic field may be provided by an external source, such as a permanent magnet or an electromagnetic coil.

Then, optionally, switch 200 may be switched back into closed mode (or, using a suitable arrangement of terminals, into an open mode), by removing or decreasing the magnetic field. It is noted that other applications for using a normally closed or normally open MEMS switch or other suitable switch in accordance with embodiments of the invention may be implemented. In alternate embodiments, a switch according to embodiments of the present invention may have other uses, and may be used in other devices. For example, such a switch may be used in other types of in-vivo devices. Further, such switches may be used in non-in-vivo devices and non-medical devices.

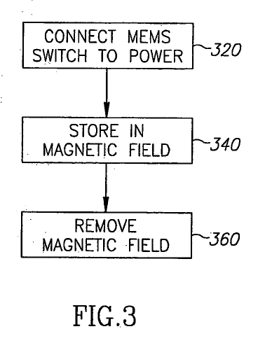

Reference is now made to FIG. 3 describing a method for keeping an in-vivo device dormant before use according to an embodiment of the present invention. In block 320 a switch such as a MEMS switch, for example, a normally open MEMS switch, may be connected, for example, electrically connected, to a power unit in an in-vivo device. During storage, the in-vivo device may be stored in the proximity of a magnetic field (340). For example, a permanent magnetic may be packaged with the in-vivo device; other suitable methods of creating a magnetic field may be implemented. The magnetic field source may be removed from the proximity of the in-vivo device (360) upon use. For example, a user may open the packaging and remove the permanent magnet. Other suitable methods may be used as well.

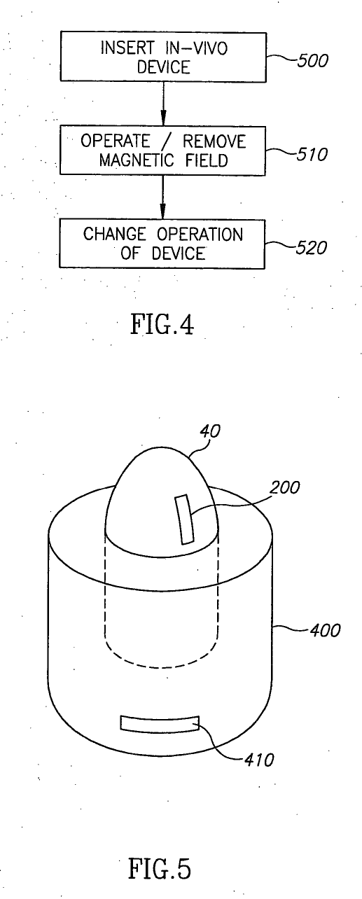

Reference is now made to FIG. 4 describing a method for altering the mode or operation of an in-vivo device according to an embodiment of the present invention. In block 500, an in-vivo device is inserted into a body, for example by swallowing, via an endoscope or other tool, etc. The in-vivo device may be as described in embodiments of the present application, or may have a different suitable structure. For example, the device may have a sensor such as an image sensor, a pH sensor, pressure sensor, etc. In block 510 an external magnetic field may operate a magnetic switch within the device. For example, an external controller may switch on or off an electromagnet, a magnet may be moved near or away from an area on a patient where the device is located, etc. The switch, depending on its structure, may become open or closed in the presence of a magnetic field. In one embodiment the switch is a normally closed MEMS switch, but other suitable switches may be used. The operation of the magnetic field may in some embodiments be in response to data (e.g., image data) received from the in-vivo. In block 520, the operation of the switch causes a mode or operation change in the device. For example, in response to the detection of a location or relative location of the in vivo device, a frame capture rate may be altered. In response to a relative lack of movement of the device, the device may lie dormant for a time, certain components (e.g., an imager, a transmitter, etc.) may have their operation reduced or stopped. Other combinations of in-vivo or device conditions and mode or operational changes may be used. Other steps or series of steps may be used.

In some embodiments a magnetic field to control a switch within an in-vivo device may be produced by a package or container designed to hold the in-vivo device, possibly during storage. Removing the device from the packaging may alter the mode of the device, for example switch the device from dormant to non-dormant mode, or turning the device on. Other mode changes may occur. Embodiments of systems and methods for packaging and a device which may be controlled by the packaging are disclosed in International Application WO 01/35813, published 25 May 2001, entitled “Method For Activating An Image Collecting Process,” incorporated by reference herein. In some embodiments, the in-vivo device may include a normally closed switch, such as a normally closed MEMS switch or another suitable switch. Such an arrangement may, in some embodiments, allow for the elimination of circuitry converting a signal from a normally open switch, reducing components, current consumption, etc. Other or different benefits may occur. FIG. 5 depicts an in-vivo device and a package or container for holding the device, according to an embodiment of the present invention. Device 40 may have a structure and operation similar to other embodiments described herein (e.g., sensor, power source, etc.), and components as described elsewhere herein are not shown in FIG. 5 for the sake of clarity. Switch 200 may be, for example, a normally closed switch, such as a MEMS switch or other suitable switch, which may when closed cause device 40 to operate and when open may cause device 40 to be inactive. Other suitable mode changes may be effected. Packaging 400 may contain or partially contain device 40 and may include a permanent magnet 410, or another suitable source of a magnetic field. Packaging 400 may be made from plastic, glass, metal or other suitable material. When device 40 is inserted in packaging 400, magnet 410 may operate switch 200 to cause switch 200 to be open, which may for example prevent the operation of device 40. When device 40 is removed from (or a certain distance from) packaging 400, switch 200 may be closed, which may for example cause or start the operation of device 40. Other suitable packaging structures may be used, and other mode changes may be effected.

While certain features of the invention have been illustrated and described herein, many modifications, substitutions, changes, and equivalents may occur to those of ordinary skill in the art. It is, therefore, to be understood that the appended claims are intended to cover all such modifications and changes as fall within the true spirit of the invention.

1. An in-vivo device comprising:
a sensor; and
a MEMS switch.

2. The in-vivo device according to claim 1 wherein the sensor is an imager.

3. The in-vivo device according to claim 1, wherein the in-vivo device is a swallowable capsule.

4. The in-vivo device according to claim 1, wherein the MEMS switch is operable to alter the mode of the device.

5. The in-vivo device according to claim 1, wherein to alter the mode of the device is in response to a magnetic field.

6. The in-vivo device of claim 1 comprising a transmitter.

7. The in-vivo device of claim 1, wherein the MEMS switch is a normally closed MEMS switch.

8. The in-vivo device of claim 1, wherein the MEMS switch comprises:
a first ferromagnetic conductive terminal;
a flexible ferromagnetic conductive terminal; and
a non-magnetic conductive terminal; wherein the first ferromagnetic conductive terminal and the non-magnetic conductive terminal are electrically isolated.

9. An in-vivo device comprising:
a sensor; and
a switch, the switch comprising:
a first ferromagnetic conductive terminal;
a flexible ferromagnetic conductive terminal; and
a non-magnetic conductive terminal; wherein the first ferromagnetic conductive terminal and the non-magnetic conductive terminal are electrically isolated.

10. The in-vivo device according to claim 9, wherein the switch is a MEMS switch.

11. The in-vivo device according to claim 9, wherein the sensor is an imager.

12. The in-vivo device according to claim 9, wherein the in-vivo device is a swallowable capsule.

13. The in-vivo device according to claim 9, wherein the switch is operable to alter the mode of the device in response to a magnetic field.

14. A system for in-vivo imaging comprising:
an in-vivo device including at least:
a sensor; and
a MEMS switch; and
an external control device, the external control device including at least a magnetic field source producing a magnetic field sufficient to operate the switch.

15. The system of claim 14, wherein the sensor is an imager.

16. The system of claim 14 comprising:
a controller to:
receive data relating to an in-vivo condition and, in response, operate the magnetic field source.

17. The system of claim 16, wherein the controller is to determine the in-vivo condition.

18. The system of claim 16, wherein the condition is the location of the in-vivo device.

19. The system of claim 14 wherein the operation of the switch alters the operation of the in-vivo device.

20. The system of claim 19, wherein the altering the operation includes stopping the operation of a component of the in-vivo device.

21. The system of claim 14, wherein the switch comprises:
a first ferromagnetic conductive terminal;
a flexible ferromagnetic conductive terminal; and
a non-magnetic conductive terminal; wherein the first ferromagnetic conductive terminal and the non-magnetic conductive terminal are electrically isolated.

22. The system of claim 14, wherein the in-vivo device is a swallowable capsule.
